(19) United States
(12) Patent Application Publication
(10) Pub. No.: US 2002/0165601 A1
(43) Pub. Date: Nov. 7, 2002

(54) Bioabsorbable stent-graft and covered stent

(76) Inventor: Claude O. Clerc, Flemington, NJ (US)

Correspondence Address: Theodore Naccarcella, Esquire, Synnestvedt & Lechner LLP, 2600 Aramark Tower, 1101 Market Street, Philadelphia, PA, 19107-2950, US

(21) Appl. No.: 09/849,141

(22) Filed: May 4, 2001

(51) Int. Cl. A61F 2/06

(52) U.S. Cl. 623/1.13

(57) ABSTRACT

The invention is a completely bioabsorbable stent-graft or covered stent. The stent preferably is a self-expanded stent of the woven or braided type made entirely of bioabsorbable material such as polylactic acid (PLA) or polyglycolic acid (PGA). The stent is either totally or partially covered by a film of a bioabsorbable material such as a bioabsorbable elastomer that can conform to the stent deformation.

### FIELD OF THE INVENTION

The invention pertains to stent-grafts and covered stents.

### BACKGROUND OF THE INVENTION

Stent-grafts and covered stents are known, respectively, for revascularization in the arterial system and for preventing tumor in-growth in ducts such as the trachea or the bronchi. Both stent-grafts and covered stents essentially comprise a support structure (the stent) covered with a porous material (in the case of a stent-graft) or non-porous material (in the case of a covered stent).

A stent essentially is a hollow tube that supplements a body lumen, such as a blood vessel. With respect to the medical condition of stenosis, in which a body lumen tends to collapse or otherwise close, the stent supports the wall of the vessel to prevent it from collapsing or closing. A blood vessel that is narrowed due to the build up of intra vascular plaque is one example of a stenosis. With respect to the medical condition of aneurism, in which a body lumen is weakened and cannot properly withstand the internal pressure within the vessel and bulges out or ruptures, the stent serves essentially the opposite function in that it supplements a weakened portion of the vessel.

Many different types of stents are commercially available at this time. Most stents need to be radially constricted, i.e., reduced in diameter, so that they can be more easily inserted into the body lumen. Once they are in situ, the stent can be radially expanded to the desired diameter. Such stents may be inserted into the body lumen in an unstressed radially minimal shape while mounted over a deflated balloon. When the stent is in situ, the balloon is inflated in order to radially expand the stent, which will then retain the radially expanded shape after the balloon is deflated and removed.

Another type of stent is termed a self-expanding stent. Self-expanding stents can be compressed radially, but will self expand to their original shape once the constricting force is removed. These designs are often made of shape memory materials, such as Nitinol, that either expand when subjected to body temperature or have superelastic properties.

Another type of self-expanding stent is a braided stent such as stent 10 shown in FIG. 1A hereof. It comprises a hollow tubular member, the wall of which is formed of a series of individual flexible thread elements 12 and 14, each of which extends helically around the central longitudinal axis of the stent. A first subset of the flexible thread elements 12 have the same direction of winding and are displaced relative to each other about the cylindrical surface of the stent. They cross a second plurality of helical thread elements 14 which are also displaced relative to each other about the cylindrical surface of the stent, but having the opposite direction of winding. Accordingly, as shown in FIG. 1A, the threads 12 of the first subset cross the threads 14 of the second subset at crossing points 16.

As the stent is axially stretched, i.e., as the longitudinal ends 18 and 20 are forced away from each other, the diameter reduces, as shown in FIG. 1B. When the force is released, the stent tends to spring back to its original diameter and length.

Artificial tubular grafts of the type relevant to this discussion are tightly woven or knitted tubes of biocompatible fabric that are used essentially to replace a damaged portion of the body, such as a damaged blood vessel. For instance, an artificial graft might be used to permanently seal a fistula or ruptured aneurysm in a blood vessel. Generally, unlike stents, grafts have a substantially fixed radius and much lower permeability than a stent. Nevertheless, grafts typically are made of knitted or woven polyethylene terephthalate (PET) yarns and are therefore porous. Another variety of graft is made out of expanded polytetrafluorethylene (ePTFE), also known a Teflon, that also is a porous structure.

Stent-grafts are medical prostheses that, as the name suggests, are essentially a combination of a stent and a graft. A stent-graft essentially provides the functions of both a stent and a graft. Particularly, the graft portion can replace a damaged portion of the vessel and substantially prevent fluid from leaving or entering the vessel through the ruptured portion while the stent portion holds the vessel open or prevents it from collapsing and also holds the stent-graft in place. Exemplary stent-grafts and covered stents are disclosed in U.S. Pat. Nos. 5,957,974, 6,156,064, 5,628,788, 5,723,004, 5,876,448 and 5,591,226, all of which are incorporated herein by reference.

A covered stent is similar to a stent-graft. The most notable difference is that a covered stent typically is nonporous.

At the time of implantation of a stent, covered stent or stent-graft (hereinafter collectively prosthesis) and in the weeks or months immediately thereafter, the prosthesis is held in position primarily by friction between the outer surface of the prosthesis and the inner surface of the body vessel that exists due to the radial expansion force of the prosthesis. Thus, the resting diameter of the prosthesis is selected to be slightly larger than the inner diameter of the vessel so that there is a constant force between the wall of the vessel and the outer surface of the stent. After a period of time, however, the tissue of the body lumen within which the stent is placed tends to grow around the stent such that it essentially becomes incorporated with the tissue of the body vessel and thus becomes permanently affixed.

Bioabsorbable stents are known in the prior art. Bioabsorbable stents are manufactured from materials that, when exposed to body fluids, dissolve over an extended period of time and are absorbed into the surrounding cells of the body. Various bioabsorbable materials that are suitable for stents are known, including polymers such as poly-L,D-lactic acid, poly-L-lactic acid, poly-D-lactic acid, polyglycolic acid, polylactic acid, polycaprolactone, polydioxanone, poly(lactic acid-ethylene oxide) copolymers, or combinations thereof. Vainionp at al., Prog Polym. Sci., vol. 14, pp. 697-716 (1989); U.S. Pat. No. 4,700,704, U.S. Pat. No. 4,653,497, U.S. Pat. No. 4,649,921, U.S. Pat. No. 4,599,945, U.S. Pat. No. 4,532,928, U.S. Pat. No. 4,605,730, U.S. Pat. No. 4,441,496, and U.S. Pat. No. 4,435,590, all of which are incorporated herein by reference, disclose various compounds from which bioabsorbable stents can be fabricated.

Partially bioabsorbable grafts also have been proposed. For instance, U.S. Pat. No. 4,997,440 discloses a vascular graft made partially of bioabsorbable materials and partially of non-absorbable material. According to that patent, the bioabsorbable component of the graft fosters increased tissue ingrowth into the graft as compared to conventional completely non-absorbable graft.

### SUMMARY OF THE INVENTION

The invention is a completely bioabsorbable stent-graft or covered stent. The stent portion preferably is a self-expanding stent of the woven, knitted or braided type made entirely of bioabsorbable material such as polylactic acid (PLA) or polyglycolic acid (PGA). The stent is either totally or partially covered by a porous or non porous film of a bioabsorbable material such as a bioabsorbable elastomer that can conform to the stent deformation.

A stent-graft in accordance with the present invention can be used where a covering is necessary to provide a scaffold for tissue ingrowth or for closing a hole such as a ruptured aneurysm or a fistula where the hole will heal itself over time and thus the need for the scaffolding is only temporary. A covered stent in accordance with the present invention can be used where a covering is necessary for closing a hole or preventing tissue in-growth, for instance, when treating a carcinoma that exerts pressure on a body duct.

### BRIEF DESCRIPTION OF THE DRAWINGS

FIG. 1A is a plan view of a braided self expanding stent in accordance with the prior art.

FIG. 1B is a plan view of the stent of FIG. 1A shown in a radially constricted/axially elongated state.

FIG. 2A is a perspective view of a stent-graft in accordance with the present invention.

FIG. 2B is an end view of a stent-graft in accordance with the present invention.

FIG. 2C is a side view of a stent-graft in accordance with the present invention in partial cut-away in order to illustrate the layers of the structure of the device.

FIG. 2D is a perspective view of an alternative embodiment of a stent=graft in accordance with the present invention.

FIG. 3 is a perspective view of a covered stent in accordance with the present invention.

FIG. 4 is a partial cut-away side view of a covered stent deployed within a blood vessel and spanning an aneurysm.

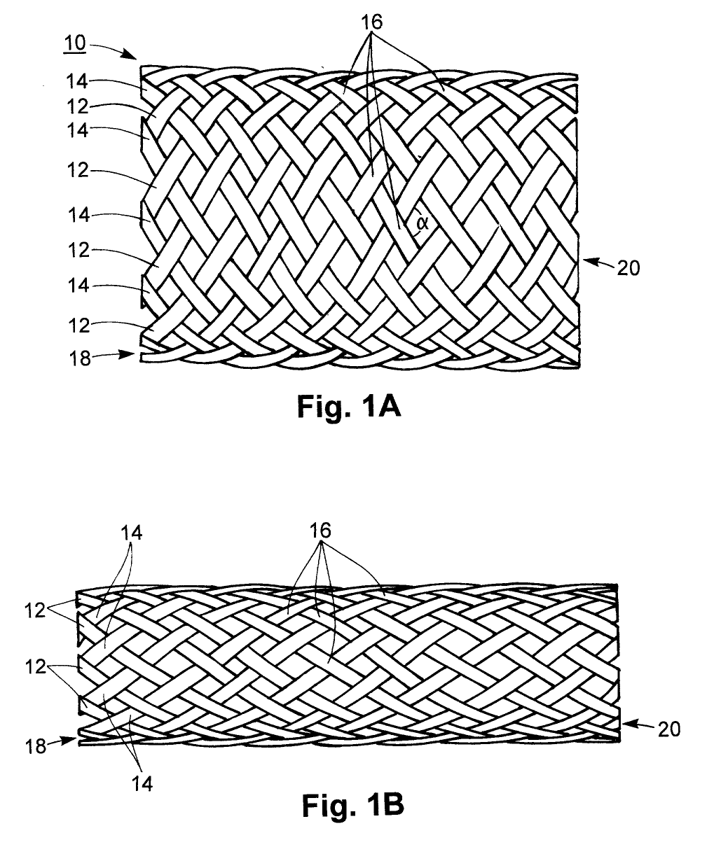

### DETAILED DESCRIPTION OF THE INVENTION

In some situations in which stent-grafts or covered stents are employed it would be the most desirable for the prosthesis to be removed after a certain period of time. For instance, an injured body vessel, such as a blood vessel, often will heal itself if a prosthesis can be implanted that will serve the function it can be supported temporarily. Therefore, it often would be desirable to provide a temporary means to support or otherwise supplement the vessel. A graft, stent-graft or covered stent can serve the above-noted functions while the vessel heals. However, after the vessel has healed and the prosthesis is no longer necessary, it remains in the body. It is highly undesirable for any prosthetic device and particularly a vascular prosthetic device to remain in the body when it no longer serves a useful function. For instance, stents, grafts, stents-grafts and covered grafts are much more prone to stenosis and thrombosis than natural vessels.

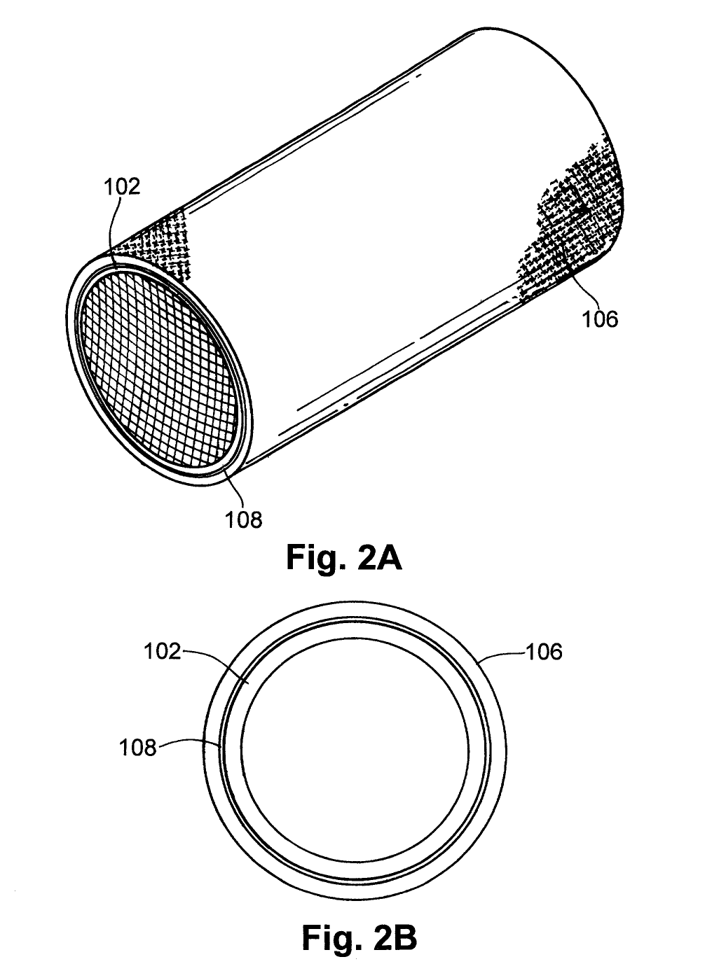

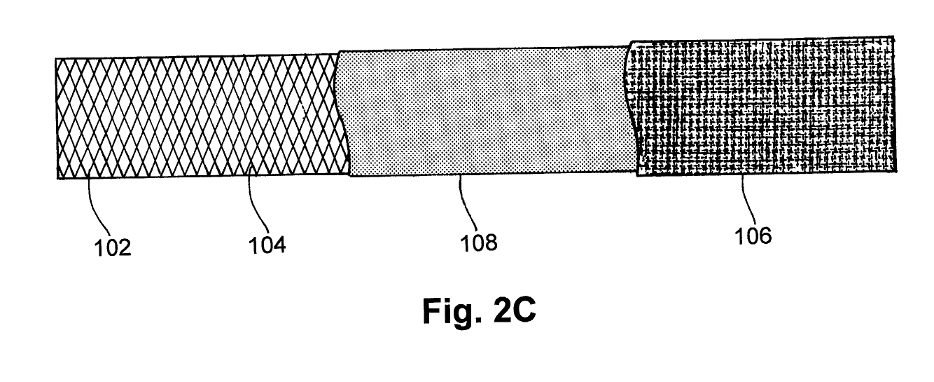

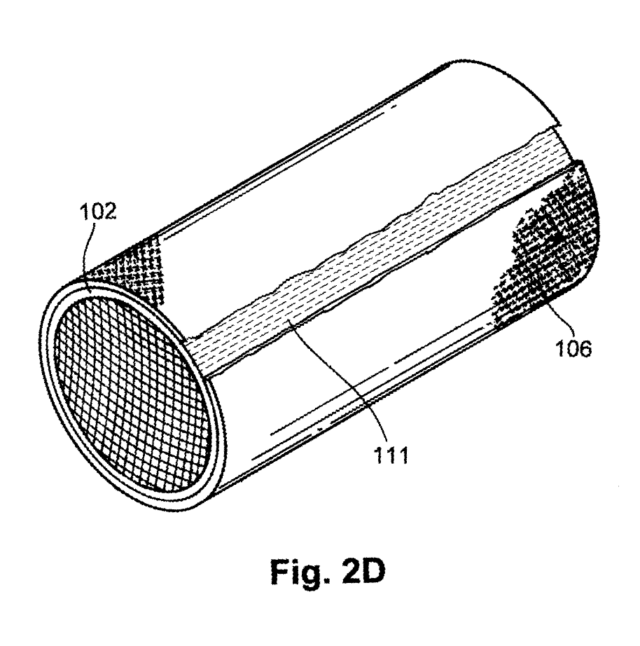

The present invention provides a stent-graft and covered stent made entirely of bioabsorbable materials such that the entire prosthesis will disintegrate over time. FIG. 2A, 2B and 2C show perspective, plan, and cut-away side views, respectively, of a bioabsorbable stent-graft 100 in accordance with the present invention. It essentially comprises three layers.

The first, innermost layer 102 is the stent structure which preferably is of a braided, knitted or woven, self-expanding, design and is made of one or more threads 104 of a bioabsorbable polymer. Various bioabsorbable materials that are suitable for stents are known, including polymers such as poly-L,D-lactic acid, poly-L-lactic acid, poly-D-lactic acid, polyglycolic acid, polylactic acid, polycaprolactone, polydioxanone, poly(lactic acid-ethylene oxide) copolymers, or combinations thereof. Vainionp at al., Prog Polym. Sci., vol. 14, pp. 697-716 (1989); U.S. Pat. No. 4,700,704, U.S. Pat. No. 4,653,497, U.S. Pat. No. 4,649,921, U.S. Pat. No. 4,599,945, U.S. Pat. No. 4,532,928, U.S. Pat. No. 4,605,730, U.S. Pat. No. 4,441,496, and U.S. Pat. No. 4,435,590, all of which are incorporated herein by reference, disclose various compounds from which bioabsorbable stents can be fabricated.

The outermost layer 106 is a porous graft layer. It can be made in accordance with any reasonable, prior art, technique. For instance, grafts typically are constructed from tightly woven, knitted or braided fabric produced by very tightly weaving, knitting or braiding one or more threads. In accordance with the present invention, however, the thread(s) are made of a bioabsorbable material, preferably a bioabsorbable elastomer and, most preferably, a bioabsorbable elastomer with elastic properties that allow it to conform to the stent deformation. Particularly, during implantation, the stent likely will be held by an insertion apparatus in an axially elongated/radially constricted shape as well known in the prior art so that the prosthesis can more easily travel through the vessel. While the graft portion of the stent-graft can be folded or otherwise collapsed in on itself in order to also reduce its diameter, it is preferable if the graft portion of the device also can be reduced in diameter consistent with the stent. Preferably, however, the axial filaments are made of a bioabsorbable elastomer. Epsilon polycaprolactone, available, for instance, from Birmingham Polymers, Inc., is a suitable bioabsorbable elastomer. Polyactive, available from Isotis, is another suitable bioabsorbable elastomer.

U.S. Pat. Nos. 5,468,253, and 5,713,920 assigned to Ethicon, Inc., describe a suitable bioabsorbable elastomer that is a copolymer of epsilon-caprolactone, trimethylene carbonate, glycolide and para-dioxanone. U.S. Pat. No. 6,113,624, also assigned to Ethicon, Inc., describes a suitable bioabsorbable elastomer that is a copolymer of lactide and p-dioxanone.

Suitable medical grade biodegradable polyurethane elastomers have also been synthesized. For instance, “Structure-Property Relationships of Degradable Polyurethane Elastomers containing an Amino Acid-Based Chain Extender” by Skarja and Woodhouse (J. Of Applied Polymer Science, Vol.75, pp. 1522-1534 (2000)) describes such biodegradable polyurethane elastomers.

Tepha, Inc., a subsidiary of Metabolix, Inc., is developing various grades of PHA (polyhdroxyalkanoate), a biocompatible and bioabsorbable polymer. The properties of these polymers range from stiff for PHB (polyhydroxybutyrate) to rubbery elastomers like PHO (polhydroxyoctanoate).

Alternately, the thread(s) of the graft layer may be formed of the same bioabsorbable polymer as the thread(s) of the stent layer, but woven in a much tighter weave.

The middle layer comprises the mechanism for attaching the graft portion to the stent portion. In the embodiment illustrated in FIGS. 2A-2C, the middle layer 108 comprises a continuous adhesive over the outer surface of the stent and the inner surface of the graft that binds them together. Most of the aforementioned bioabsorbable polymers out of which the elastomeric graft may be made also would be suitable for the adhesive. Particularly, the polymer can be dissolved in a solvent and used as the adhesive. One or both of the layers can be covered with the solution and the two layers can be brought into contact. The solvent can then be evaporated by heat treating the stent-graft, leaving behind the polymer layer 108 binding the inner layer 102 and outer layer 104 to each other.

In certain instances, particularly those instances where the graft layer does not conform to the stent deformation, it may be difficult to attach the graft layer to the stent layer with a continuous layer of adhesive. Accordingly, the graft may be attached to the stent by adhesive only at intervals. In one embodiment, a series of longitudinal bands of adhesive or sutures joining a strip of the outer surface of the stent to a strip of the inner surface of the graft may be employed. In this manner, the portion of the graft that are not adhered or otherwise attached to the stent can fold when the stent is radially constricted.

Other alternatives for attaching the stent layer and the graft layer include heat sealing. For instance, the graft and stent layers can be mated while mounted on a mandrel and the prosthesis can be heated above the melt temperature of one or both of the graft layer polymer and the stent layer polymer to cause them to heat seal to each other. Even further, the stent threads can be coated with a bioabsorbable polymer (by spraying or dipping in solution) and the graft layer can be attached to the stent as just described. Even further, the two layers can be laminated to each other in any well known manner. Even further, two graft layers can be employed wherein one graft layer is laid over the stent layer and the other is laid within the stent layer and the two graft layers are laminated to each other through the stent layer, such as by heating the prosthesis above the melt point of the graft layer polymer so that the two layers heat seal to each other.

In an even further alternate embodiment illustrated in FIG. 2D, the graft layer and the stent layer may be attached by bioabsorbable sutures 111 which can be formed of the same material as the threads of the stent or the graft layer. FIG. 2D is a partially cutaway perspective view of an exemplary stent-graft similar to the stent-graft of FIGS. 2A-2C, except that, instead of an adhesive layer 108, there are a plurality of bioabsorbable sutures 111 that hold the stent and graft layers together.

While the stent-graft of the present invention has been discussed herein above in connection with an embodiment in which the graft layer surrounds the stent layer, in other embodiments, the graft layer can be inside the stent layer.

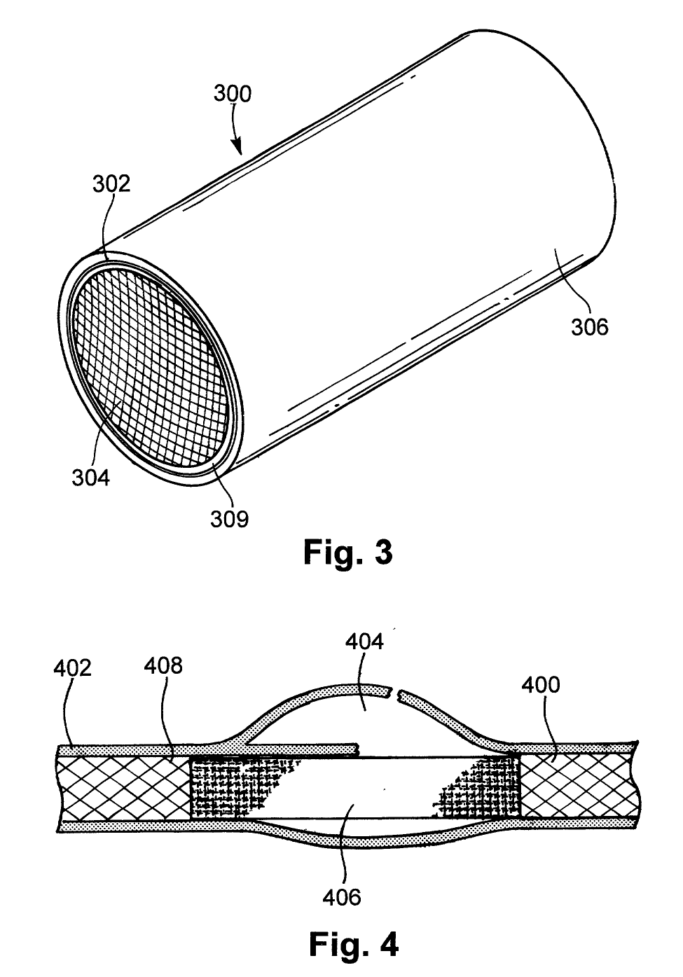

FIG. 3 shows a covered stent 300 in accordance with the present invention. It is essentially similar to the stent-graft illustrated in FIGS. 2A-2C and discussed above except that the outermost layer is non-porous.

As discussed above in connection with the stent graft embodiment of the invention, the first, innermost layer 302 is the stent structure which preferably is of a braided or woven, self-expanding, design and is made of one or more threads 304 of a bioabsorbable polymer, such as any of the polymers mentioned above in connection with the stent layer of the stent-graft embodiment of the invention.

The outermost layer 306 is a non-porous polymer layer. It can be made in accordance with any reasonable, prior art, technique for manufacturing non-porous stent covers. For instance, a continuous film of any of the aforementioned bioabsorbable polymer materials can form the layer 306. Various ways to manufacture a continuous film of a polymer would be apparent to persons of skill in the art of polymer processing.

The cover can be folded or otherwise collapsed in on itself during insertion, when the stent is in the radially contracted state. If the cover is made of a bioabsorbable elastomer, such as aforementioned epsilon caprolactone, polyactive, polyhdroxyalkanoate, or other polyurethane based biodegradable elastomers, it may also have some ability to be reduced in size during insertion.

The cover 306 can be attached to the stent portion 302 in any of the ways discussed above in connection with the stent-graft embodiment of the invention, including adhesive 309. If a suture or other mechanical type of attachment is used, it should be assured that the mechanical attachment mechanism does not create openings in the cover layer that would compromise the non-porosity of the cover layer. For instance, if the cover layer 306 is sutured to the stent layer 302, the prosthesis may be heat treated to melt the suture and/or cover layer so as to seal any gaps between the sutures and the cover where the sutures pass through the cover. Of course, in many applications, maintenance of total non-porosity may not be necessary and such steps may be unnecessary.

While the covered stent of the present invention has been discussed herein above in connection with an embodiment in which the graft layer overlays the stent layer, in other embodiments, the cover layer can be inside the stent layer.

FIG. 4 illustrates a stent-graft 400 deployed in a blood vessel 402 and spanning an aneurysm 404. The stent-graft graft 400 both supports the vessel to keep it from collapsing (by virtue of the stent layer 406) and substantially seals the aneurysm (by virtue of the graft layer 408) to keep blood from leaking out of the vessel or other bodily fluids form entering the vessel through the rupture.

There are several additional methods for manufacturing stent-grafts and covered stents in accordance with the present invention. For instance, a stent body can be fabricated in any of the known techniques. In the case of a covered stent, it can then be coated with a non-porous covering by dipping the stent, with or without a mandrel inside the stent, in a solution comprising a bioabsorbable polymer dissolved in as solvent. The solvent can then be evaporated from the solution in a heat treatment process thus leaving a continuous film of the bioabsorbable polymer on the stent layer. Preferably, during the heat treatment step, the prosthesis is positioned with its longitudinal axis horizontal and the prosthesis is rotated about its longitudinal axis to obtain a consistent thickness of the cover material.

In another embodiment, a stent body can be manufactured in accordance with any known technique and the stent body sprayed with a solution of the bioabsorbable polymer in solvent. Again, the prosthesis is then heat treated to evaporate the solvent portion of the solution, thus leaving a coating of the bioabsorbable polymer on the stent.

In the case of a porous stent-graft, a porous coating can be made essentially as described above but with the addition of salt crystals in the solution (or any other particulate substance that is not soluble in the solution). The salt crystals will be embedded in the stent-graft during the dipping (or spraying) step. After the heat treating step, the prosthesis can be dipped in a liquid within which the salt crystals will dissolve, but which will not affect the bioabsorbable polymer coating, thereby leaving openings in the coating.

In yet a further embodiment, a porous coating could be provided by covering the stent body with adhesive, such as by dipping or spraying, and laying threads on the stent body in a regular or random pattern.

A stent-graft or covered stent manufactured in accordance with the present invention will be entirely bioabsorbable and can be used where a covering is necessary to provide a scaffold for tissue ingrowth or for temporarily closing a rupture such as a fistula.

In further embodiments of the invention, the cover or graft layer can have a drug incorporated into it so that the drug is released as bioabsorption of the cover or graft layer occurs.

Further, any or all of the bioabsorbable materials can be made radiopaque by the addition of a radiopaque filler, such as a metal or ceramic powder, during fabrication of the threads or film.

Even further, axial runners, particularly bioabsorbable axial runners, can be incorporated into the stent body in order to provide enhanced radial expansion force. U.S. patent application Ser. No. 09/626,638 entitled “Self Expanding Stent with Enhanced Radial Expansion and Shape Memory”, filed on Jul. 27, 2000 and owned by the assignee of the present invention as well as U.S. patent application Ser. No. ______ entitled “Method for Attaching Axial Fibers to a Self-Expanding Stent and Self-Expanding Stent with Axial Fibers” (Attorney Docket No. 24676 USA) also assigned to the assignee of the present application disclose stents with axial runners and methods of attaching the axial runners to the stent body. Both of those patent applications are incorporated herein by reference.

While the embodiments of FIGS. 2A-2C and 3 show the graft material or covering material as covering the entire length of the stents, this is not necessary. The covering material can cover any portion of the stent.

The threads composing the stents can be coated with a binding agent to enhance the adhesion of the graft or covering layer.

Having thus described a few particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

1. A bioabsorbable tubular prosthesis comprising:
a fully bioabsorbable first stent layer for supporting a body vessel; and
a fully bioabsorbable second layer attached to said first layer for substantially sealing said body vessel.

2. The prosthesis of claim 1 wherein said first layer forms a self expanding tube and comprises at least one braided, knitted or woven thread.

3. The prosthesis of claim 2 further comprising a third layer comprising adhesive interposed between said first and second layers.

4. The prosthesis of claim 2 wherein said tubular prosthesis has a longitudinal axis and said first and second layers are attached to each other by a plurality of bands of adhesive interposed between said first and second layers.

5. The prosthesis of claim 4 wherein said bands of adhesive are parallel to said longitudinal axis of said prosthesis.

6. The prosthesis of claim 2 wherein said first and second layers are attached to each other by bioabsorbable sutures.

7. The prosthesis of claim 2 wherein said second layer is heat sealed to said first layer.

8. The prosthesis of claim 1 wherein said second layer is porous.

9. The prosthesis of claim 1 wherein said prosthesis is a stent-graft.

10. The prosthesis of claim 9 wherein said second layer is formed of at least one tightly woven, knitted or braided thread of bioabsorbable polymer.

11. The prosthesis of claim 1 wherein said second layer is non-porous.

12. The prosthesis of claim 11 wherein said second layer comprises a sheet of bioabsorbable polymer.

13. The prosthesis of claim 1 wherein said prosthesis is a covered stent.

14. The prosthesis of claim 2 wherein said second layer is comprised of a bioabsorbable elastomer.

15. A method of manufacturing a bioabsorbable tubular prosthesis comprising a fully bioabsorbable first stent layer for supporting a body vessel and a fully bioabsorbable second layer attached to said first layer for substantially sealing said body vessel, said method comprising the steps of:
(1) providing a stent body;
(2) providing a layer comprised entirely of bioabsorbable material;
(3) attaching said layer to said stent body.

16. The method of claim 15 wherein said stent body is formed of at least one braided, woven or knitted thread.

17. The method of claim 16 wherein step (3) comprises adhering said layer to said stent body with a bioabsorbable adhesive.

18. The method of claim wherein step (3) comprises:
(3.1) dissolving a bioabsorbable polymer in a solvent to create a solution;
(3.2) applying said solution to said stent body;
(3.3) bringing said layer in contact with said stent body and said solution; and
(3.4) applying heat to evaporate said solvent.

18. The method of claim 15 wherein step (3) comprises:
(3.1) bringing said layer in contact with said stent body and said solution; and
(3.2) heating said prosthesis above a melt temperature of at least one of said stent body and said layer so as to heat seal said layer to said stent body.

19. The method of claim 15 wherein step (3) comprises:
(3.1) mechanically attaching said layer to said stent body with bioabsorbable sutures.

20. A method of manufacturing a bioabsorbable tubular prosthesis comprising a fully bioabsorbable first stent layer for supporting a body vessel and a fully bioabsorbable second layer attached to said first layer for substantially sealing said body vessel, said method comprising the steps of:
(1) providing a stent body;
(2) covering said stent body with a solution comprising a bioabsorbable polymer dissolved in a solvent; and
(3) heating said prosthesis to dissolve said solvent, whereby a coat of said bioabsorbable polymer is left on said stent body.

21. The method of claim 20 wherein step (2) comprises dipping said stent body in said solution.

22. The method of claim 20 wherein step (2) comprises spraying said stent body with said solution.

23. The method of claim 20 wherein said solution further comprises salt crystals dissolved in said solvent whereby said salt crystals are embedded in said coating and wherein said method further comprises the steps of:
(4) after step (3), dissolving said salt crystals to leave openings in said coating.

24. The method of claim 23 further comprising the step of:
(5) rotating said stent body during said step (3).

25. A method of manufacturing a bioabsorbable tubular prosthesis comprising a fully bioabsorbable first stent layer for supporting a body vessel and a fully bioabsorbable second layer attached to said first layer for substantially sealing said body vessel, said method comprising the steps of:
(1) providing a stent body;
(2) covering said stent body with an adhesive; and
(3) laying bioabsorbable threads in said adhesive on said stent body to form said bioabsorbable second layer.
